(19) United States
(12) Patent Application Publication
(10) Pub. No.: US 2010/0160995 A1
(43) Pub. Date: Jun. 24, 2010

(54) METHOD FOR TREATING OBESITY

(76) Inventor: Jerome DARGENT, Lyon (FR)

Correspondence Address: CANTOR COLBURN, LLP, 20 Church Street, 22nd Floor, Hartford, CT, 06103, US

(21) Appl. No.: 12/338,287

(22) Filed: Dec. 18, 2008

(51) Int. Cl. A61N 1/36; A61B 17/00

(52) U.S. Cl. 607/40; 128/898

(57) ABSTRACT

Method for treating obesity in a mammal which has an oesophagus connected to a stomach at the level of an oesophagogastric junction region, the oesophagogastric junction region exhibiting a zigzag oesophagogastric mucosal junction line and a transverse mucosal fold raised by sling fibres, the mucosal fold being located downstream of the Z line, the mucosal fold and the Z line defining a conical structure delimiting an opening to the stomach,

including creating a first stricture zone by reducing the cross section of the opening by introducing at least a first biocompatible substance implanted on or in the wall of the conical structure, at least at the level of the mucosal fold.

The present invention relates to a method for treating obesity by reducing the opening, or calibre, of the digestive tube as it enters the stomach, delimited on the one hand by the oesophagogastric mucosal junction Z line and on the other hand by the sling fibres of Helvetius in the anatomical region of the oesophagogastric junction.

For a long time, most of the treatments for morbid obesity have required an extensive surgical procedure. Among these treatments, mention may be made of implanting a gastric band, intended to compress the stomach by surrounding it and to thus produce an external restriction of the stomach, so as to reduce the capacity of the latter to absorb food, or else performing a gastric by-pass consisting in creating a gastric pocket directly connected to the intestine.

Although these procedures have become easier for the surgeon and less restrictive for the patient with the emergence and the generalization of laparoscopy techniques, they remain surgical procedures with major drawbacks linked to one another: the cost and the extensive nature of a procedure, the constant ethical choice of dealing only with a limited population suffering from morbid obesity, the psychological barrier in patients who fear the consequences of a surgical procedure, the difficulty in modulating the effects of these procedures.

It is therefore important to seek to develop methods for treating obesity which are less invasive, preferably ambulatory, thus requiring only short anaesthesia or alternatively simple analgesia, making it possible to treat the patient during the day for example, without a surgical procedure.

Currently, there exists mainly a non invasive technique for treating obesity, this technique consisting in introducing an inflatable balloon into the stomach in order to reduce the size of the latter, for a limited period of time, for example six months. The patient thus reaches the feeling of satiety more rapidly. However, the effectiveness of this technique is relative and short term, the patient generally putting weight back on once the balloon has been removed.

Thus, there exists the need for a non invasive, effective, modulatable method for treating obesity which is simple to carry out and which would make it possible to rapidly treat all patients suffering from obesity, whether or not this obesity is morbid, i.e. with a BMI (body mass index) of greater than 35.

The present invention is directed towards remedying this need by proposing a method for treating obesity which in particular can be carried out entirely endoscopically, rapidly and simply, it also being possible to modulate the effects of said method according to the patient's response.

A subject of the present invention is a method for treating obesity in a mammal which has an oesophagus connected to a stomach at the level of an oesophagogastric junction region, said oesophagogastric junction region exhibiting a zigzag oesophagogastric mucosal junction line and a transverse mucosal fold raised by sling fibres, said mucosal fold being located downstream of said Z line, said mucosal fold and said Z line defining a conical structure delimiting an opening to the stomach, comprising at least the following step:

a°) a first stricture zone is created by reducing the cross section of said opening by introducing at least a first biocompatible substance implanted on or in the wall of said conical structure, at least at the level of said mucosal fold.

The method according to the invention does not require any surgical procedure which is traumatic for the flesh, nor any sutures. The entire procedure can be carried out via the natural routes, such as the mouth, the oesophagus and the stomach. The method according to the invention is simple and rapid for the surgeon to perform. Since the procedure does not require deep anaesthesia with intubation, it is possible to treat the patient during the day, the patient being able to go home immediately after the procedure.

Moreover, according to the method of the invention, it is possible to control the size of the reduction in cross section obtained and to thus adjust this reduction to the specific needs of a given patient. It is thus possible to treat both patients suffering from a low level of obesity and patients suffering from morbid obesity.

In a preferred embodiment of the invention, said first biocompatible substance is implanted endoscopically using at least one endoscope.

Preferably, said first biocompatible substance is implanted by circumferential injection, under the mucosa of said wall or in a muscle layer of said wall, of a polymer which solidifies after injection. For example, said first biocompatible substance is injected at four distinct sites, located on the same circumference. Said first biocompatible substance may be bioresorbable or nonbioresorbable. As bioresorbable biocompatible substances suitable for the method according to the invention, mention may be made of collagen and hyaluronic acid, and mixtures thereof. Advantageously, said polymer is nonbioresorbable. As nonbioresorbable biocompatible substances, mention may be made of ethylene/vinyl alcohol copolymers, such as the product sold under the trade name “Enteryx®” by the company Boston Scientific Corporation, or else hydrogels based on polyacrylonitrile polymers, and mixtures thereof. Such circumferential injections are known in the endoscopic treatment of gastro-oesophageal reflux and so are concerned only with the oesophagogastric mucosal junction Z line. Such an injection technique and also nonbioresorbable polymers that are particularly suitable for the method according to the invention are, for example, described in document U.S. Pat. No. 6,238,335.

In one embodiment of the invention, prior to step a°), the implantation site is defined by retrovision, by turning round said endoscope that was previously introduced into the stomach. In fact, the pinpointing of the implantation site, in particular at the level of the conical structure, is a delicate step in the method according to the invention. If the injection of polymers is carried out too far upstream of the conical structure, it becomes virtually oesophageal and loses all its effectiveness. On the other hand, if this injection is carried out too far downstream of this conical structure, it has little chance of becoming circular and the creation of a stricture zone will prove to be virtually impossible. In the present application, the term “region located upstream of the conical structure” is intended to mean the region located between the conical structure and the mouth, and the expression “region located downstream of the conical structure” is intended to mean the region located between the conical structure and the stomach. Thus, the injection site is difficult to pinpoint by direct vision, the gastric mucosa appearing to vanish once the oesophagogastric mucosal junction Z line has been crossed. Moreover, in the case of a hiatus hernia, which is very common in obese individuals, this assessment is even more difficult. The pinpointing of the zone to be implanted, by retrovision, gives an appropriate panoramic view.

In one embodiment of the invention, the method according to the invention also comprises an additional step b°) chosen from i) a second reduction of the cross section of said opening by means of a further implantation of a second biocompatible, optionally bioresorbable, substance, on or in the wall of said conical structure, ii) the addition of an internal mucosal plicature upstream of said first stricture zone, produced either by suturing or by means of an internal stapling device, iii) the implantation, preferably prior to said step a°), of a gastric balloon, iv) a partitioning of the stomach, for instance a sleeve gastrectomy or a gastrojejunal bypass, v) a gastric electrical stimulation, or the combination of one or more of these steps.

In particular, when step b°) consists in performing a second reduction of the cross section of said opening, an implantation of a second biocompatible substance upstream of said first stricture zone may be added. Alternatively, the entire wall of said conical structure may be covered with a second biocompatible substance. It is thus possible, if desired, to accentuate the effect of the stricture obtained in step a°). Said second biocompatible substance may or may not be identical to said first biocompatible substance. In a preferred embodiment, said second biocompatible substance is bioresorbable. This method has the advantage of making it possible to very easily go back in the event of accentuated dysphasia, by simple endoscopic dilation. A technique for the addition of a plicature that may be suitable for the method according to the present invention is, for example, described in document U.S. Pat. No. 6,238,335. Similarly, nonbioresorbable polymers and bioresorbable polymers, forming the first and second biocompatible substances, that are particularly suitable for the method according to the invention are, for example, described in document U.S. Pat. No. 6,238,335.

In another embodiment of the invention, when step b°) consists in performing the addition of an internal mucosal plicature upstream of said first stricture zone, a mini-pocket, built against said first stricture zone, is created by means of sutures, said pocket being renewable on demand. Such a step makes it possible to ensure a compartmentalization creating a real satiety. As previously, this technique has the advantage of making it possible to very easily go back in the event of accentuated dysphasia, by simple endoscopic dilation.

In yet another embodiment of the invention, when step b°) consists in performing a gastric electrical stimulation, a bipolar electrode is implanted laparoscopically into the antral gastric muscularis, in the region of the nerves of the pes anserinus, said electrode being connected to a subcutaneous stimulation housing delivering electric pulses having effects on gastric distension and the gastric stretch receptors of the muscularis, satiety after meals and appetite between and before meals.

Such a gastric stimulation technique is known. The addition of this technique to the reduction in cross section of the opening obtained according to step a°) of the method according to the invention potentiates, and at the very least significantly initiates, weight loss. This technique also makes it possible to modulate the effects of the method according to the invention, the electrical stimulation parameters being adjustable.

The endoscopes and ancillary equipment suitable for putting the method according to the invention into practice are known tools suitable, for example, for the endoscopic treatment of gastro-oesophageal reflux, such as those described in document U.S. Pat. No. 6,238,335.

In another embodiment of the invention, when step b°) consists of the implantation of a gastric balloon, step b°) is preferably carried out before step a°), one after the other: thus, a gastric balloon is implanted in the stomach of a patient, in order to mechanically reduce the size of the stomach. After a few months, the gastric balloon is removed from the patient's stomach and step a°) of the method according to the invention is carried out at the time the gastric balloon is removed. Step a°) according to the invention thus prolongs the effect of the gastric balloon in a non-traumatic manner.

In another embodiment of the invention, when step b°) consists of a partitioning of the stomach, for instance a sleeve gastrectomy or a gastrojejunal bypass, the techniques of the two steps a°) and b°) can be combined at the same time, one supplementing the effects of the other.

Other advantages of the present invention will emerge from the detailed description which follows and from the attached drawings in which:

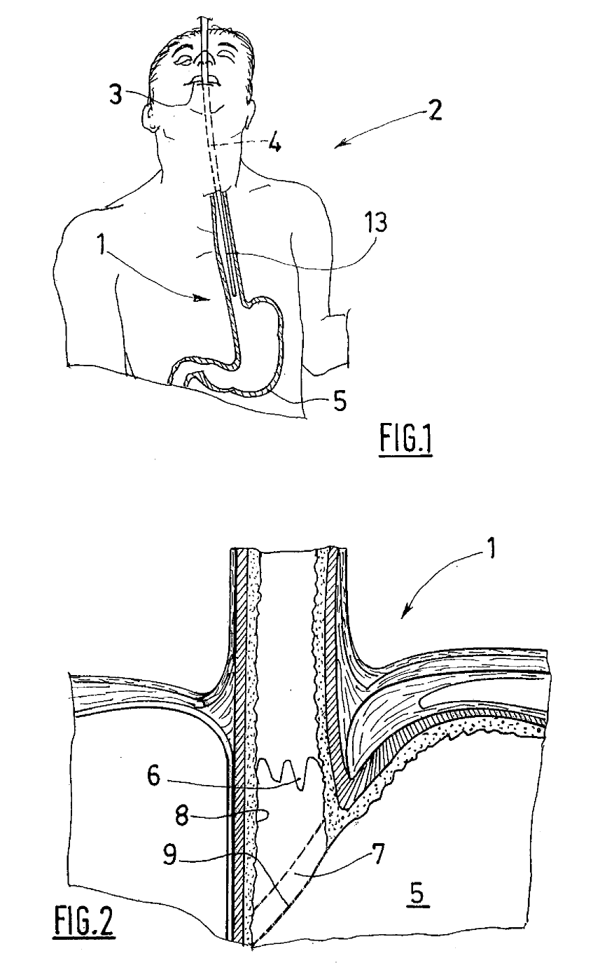

FIG. 1 is a schematic view of the region of the oesophagogastric junction in a human,

FIG. 2 is a sectional view of the oesophagogastric region showing the conical structure targeted by the method according to the invention,

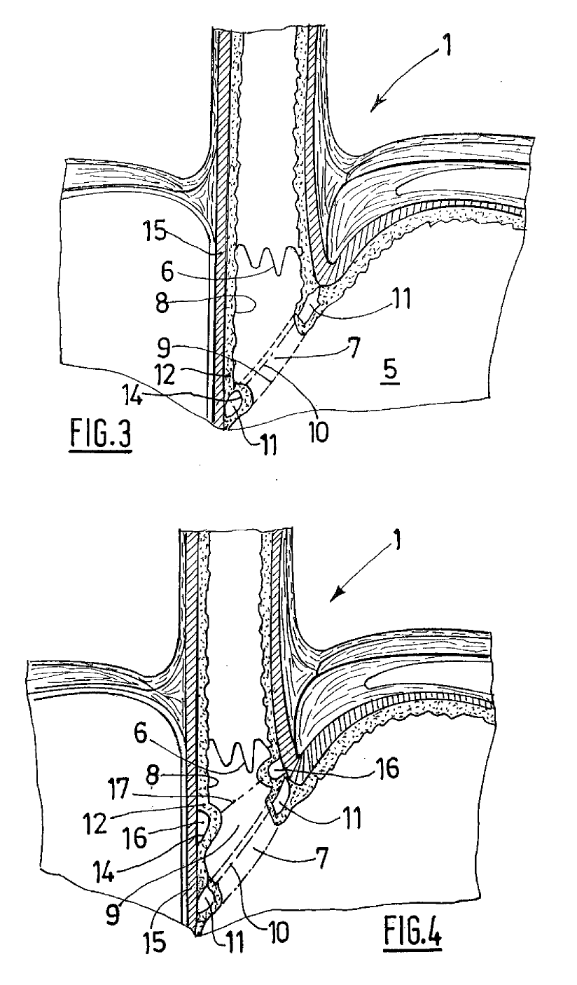

FIG. 3 is a sectional view of the region of FIG. 2 once the first biocompatible substance has been implanted,

FIG. 4 is a sectional view of the region of FIG. 2 once a double reduction of the cross section of the opening has been performed,

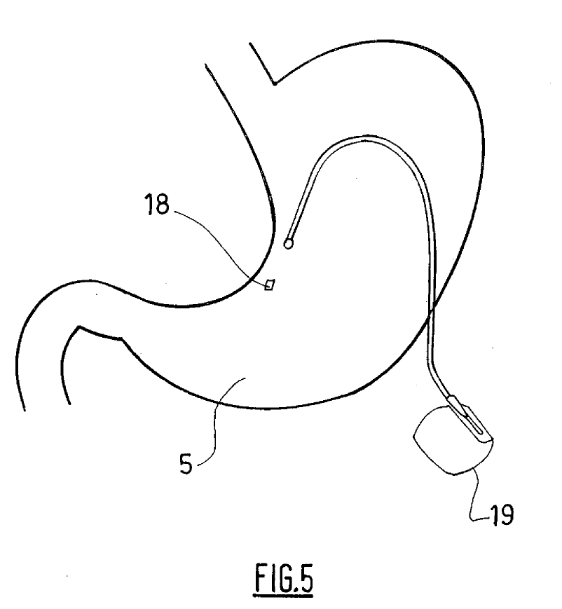

FIG. 5 is a side view of a stomach in which a bipolar electrode has been implanted for gastric stimulation according to a step b°) of the method according to the invention.

Represented in FIG. 1 is the oesophagogastric region 1 of a human being 2 comprising a mouth 3, an oesophagus 4 and a stomach 5. As can be seen more clearly on FIG. 2, the oesophagogastric region 1 has an oesophagogastric mucosal junction Z line 6 and a transverse mucosal fold 7 raised by sling fibres. The mucosal fold 7 and the Z line define a conical structure 8 delimiting an opening 9 to the stomach 5.

As can be seen in FIG. 3, a first stricture zone 10 is created, according to the method of the invention, by reducing the cross section of the opening 9 by introducing at least a first biocompatible substance 11 implanted on or in the wall 12 of said conical structure 8 at the level of the mucosal fold 7.

Preferably, the first biocompatible substance 11 is implanted endoscopically using an endoscope 13 as shown in FIG. 1.

On the example represented in FIG. 3, the first biocompatible substance 11 is implanted under the mucosa 14 of the wall 12. In one embodiment of the invention not represented, the first biocompatible substance 11 is implanted in a muscle layer 15 of the wall 12. The first biocompatible substance 11 is implanted by circumferential injection of a polymer which solidifies after injection. For example, this polymer is injected at four distinct sites located on the same circumference. Preferably, this polymer is nonbioresorbable.

Preferably, the implantation site is defined by retrovision, by turning round the endoscope 13 that was previously introduced into the stomach 5.

Represented in FIG. 4 is the oesophagogastric region of FIG. 3, to which has been added an additional step of reduction of the opening 9 by means of a further implantation of a second biocompatible substance 16, which may or may not be identical to the first biocompatible substance 11 implanted at the level of the mucosal fold 7, it being possible for this second biocompatible substance 16 to be optionally bioresorbable and this second biocompatible substance 16 being implanted on or in the wall of said conical structure 8, upstream of said first biocompatible substance 11, i.e. upstream of the first stricture zone 10. The implantation of this second biocompatible substance has the effect of creating a second stricture zone 17 upstream of the first stricture zone 10.

Alternatively, according to one nonrepresented embodiment of the invention, a second reduction of the cross section of the opening 9 is performed by covering the entire wall 12 of the conical structure 8 with a second biocompatible substance 16.

In another nonrepresented embodiment, said second biocompatible substance 16 is implanted over said first biocompatible substance, at the level of the mucosal fold 7, in order to further reduce the first stricture zone 10.

According to another nonrepresented embodiment of the invention, the addition of an internal mucosal plicature upstream of said first stricture zone 10 is performed, creating, by means of sutures, a mini-pocket built against said first stricture zone 10, said pocket being renewable on demand.

According to another nonrepresented embodiment of the invention, the addition of an internal mucosal plicature is carried out by means of internal stapling or gastric endostapling: in such a case, it is possible to create the stricture zone 10 according to the invention at the level of the bottom (or end) part of the stapling, which may be located slightly downstream of the conical structure 8.

According to another nonrepresented embodiment of the invention, the addition of a “conventional” gastric plicature is carried out by laparoscopy, such as a gastrojejunal bypass or alternatively a sleeve gastrectomy.

According to another nonrepresented embodiment of the invention, a gastric balloon is initially implanted in the stomach of a patient. After a few months, for example six months, the gastric balloon is removed from the patient's stomach and the stricture zone is created according to the method of the present invention. The stricture zone created in this way thus prolongs the effect of the gastric balloon.

According to yet another embodiment of the invention, as shown in FIG. 5, a gastric electrical stimulation is performed by implanting a bipolar electrode 18 within the antral gastric muscularis of the stomach 5, said electrode 18 being connected to a subcutaneous stimulation housing 19 delivering electric pulses having effects on gastric distension and the gastric stretch receptors of the muscularis, satiety after meals and appetite between and before meals.

Generally, the electrode 18 is implanted laparoscopically, preferably in the region of the nerves of the pes anserinus of the stomach.

The method according to the invention is particularly simple, can be carried out rapidly and does not require any deep anaesthesia or extensive surgical procedure. It may be suitable for any patient suffering from a low or medium level of obesity or morbid obesity. It is not traumatic for the flesh and can be easily modulated and combined with other supplementary noninvasive techniques.

1. Method for treating obesity in a mammal which has an oesophagus connected to a stomach at the level of an oesophagogastric junction region, said oesophagogastric junction region exhibiting a zigzag oesophagogastric mucosal junction line and a transverse mucosal fold raised by sling fibres, said mucosal fold being located downstream of said Z line, said mucosal fold and said Z line defining a conical structure delimiting an opening to the stomach, comprising
a first stricture zone is created by reducing the cross section of said opening by introducing at least a first biocompatible substance implanted on or in the wall of said conical structure, at least at the level of said mucosal fold.

2. Method according to the claim 1, wherein said first biocompatible substance is implanted endoscopically using at least one endoscope.

3. Method according to claim 1, wherein said first biocompatible substance is implanted by circumferential injection, under the mucosa of said wall or in a muscle layer of said wall, of a polymer which solidifies after injection.

4. Method according to claim 3, wherein said polymer is nonbioresorbable.

5. Method according to claim 2, wherein, prior to step a°), the implantation site is defined by retrovision, by turning round said endoscope which was previously introduced into the stomach.

6. Method according to claim 1, further comprising an additional step b°) chosen from i) a second reduction of the cross section of said opening by means of a further implantation of a second, optionally bioresorbable, biocompatible substance, on or in the wall of said conical structure, ii) the addition of an internal mucosal plicature upstream of said first stricture zone, performed either by suturing or by means of an internal stapling device, iii) the implantation, preferably prior to said step a°), of a gastric balloon, iv) a partitioning of the stomach, for instance a sleeve gastrectomy or a gastrojejunal bypass, v) a gastric electrical stimulation, or the combination of one or more of these steps.

7. Method according to claim 6, wherein, since step b°) consists in performing a second reduction of the cross section of said opening, the entire wall of said conical structure is covered with a second biocompatible substance.

8. Method according to claim 6, wherein, since step b°) consists in performing a second reduction of the cross section of said opening, an implantation of a second biocompatible substance upstream of said first stricture zone is added.

9. Method according to claim 6, wherein, since step b°) consists in performing the addition of an internal mucosal plicature upstream of said first stricture zone, a mini-pocket built against said first stricture zone is created, by means of sutures, said pocket being renewable on demand.

10. Method according to claim 6, wherein, since step b°) consists in performing a gastric electrical stimulation, a bipolar electrode is implanted laparoscopically within the antral gastric muscularis, in the region of the nerves of the pes anserinus, said electrode being connected to a subcutaneous stimulation housing delivering electric pulses having effects on gastric distension and the gastric stretch receptors of the muscularis, satiety after meals and appetite between and before meals.
